(19) United States
(12) Patent Application Publication
(10) Pub. No.: US 2024/0215910 A1
(43) Pub. Date: Jul. 4, 2024

(54) A METHOD FOR EVALUATING THE PRO-OR ANTI CONVULSIVE PROPERTIES OF TEST COMPOUNDS

(71) Applicant: GW Research Limited, Cambridgeshire (GB)

(72) Inventors: Karen Ka-Yen TSE, Cambridgeshire (GB); Royston Anthony GRAY, Cambridgeshire (GB)

(21) Appl. No.: 18/257,479

(22) PCT Filed: Dec. 8, 2021
(86) PCT No.: PCT/GB2021/053204

§ 371 Date: Jun. 14, 2023

(30) Foreign Application Priority Data
Dec. 15, 2020 (GB) 2019787.7

(51) Int. Cl. A61B 5/00; A61B 5/11; G01N 33/94

(52) U.S. Cl. CPC A61B 5/4848; A61B 5/1107; A61B 5/4094; G01N 33/9473; A61B 2503/40

(57) ABSTRACT

The present invention relates to a method of evaluating the pro- or anti convulsive properties of test compounds that is both streamlined and is capable of providing a clear indication for the selection of candidate compounds during preclinical assessment.

### FIELD OF THE INVENTION

The present invention relates to a method of evaluating the pro- or anti convulsive properties of test compounds that is both streamlined and is capable of providing a clear indication for the selection of candidate compounds during preclinical assessment.

### BACKGROUND TO THE INVENTION

In the routine laboratory screening of new antiepileptics, the selection of appropriate animal models for the initial in vivo testing of potential anticonvulsant compounds is a highly important decision in the successful search for new antiepileptic drugs.

The standard maximal electroshock seizure threshold (MEST) test is widely utilized preclinically to evaluate pro- or anti-convulsive properties of test compounds (Löscher et al., 1991). The MEST test is typically conducted in rodents. An increase in seizure threshold is indicative of an anticonvulsive effect. Antiepileptic drugs such as sodium valproate, which has clinically proven efficacy against generalised tonic-clonic seizures, has been shown to have anticonvulsive properties in this test in the mouse. Conversely, a reduction in seizure threshold is indicative of a proconvulsive effect as observed with known convulsive agents (e.g. Picrotoxin).

The ability of a test compound to alter the stimulus intensity, expressed as current (mA), required to induce the presence of tonic hind limb extensor convulsions, is assessed in the MEST. The outcome of the presence (+) or absence (0) of tonic hind limb extensor convulsions observed from a current to produce tonic hind limb extension in 50% of animals in the treatment group (CC₅₀) determines the seizure threshold for the treatment group; the effects are then compared to the CC₅₀ of a vehicle control group.

Disadvantages of MEST test are that a high number of animals have to be used (at least 12) in order to verify the result, this in turn means that a large amount of test compound is required in order to dose all the animals at the desired doses.

For many years it has been a principle of preclinical research to find ways to limit or substitute the use of animals for drug screening. The so called 4R's of research are defined as Reduction, Refinement, Replacement and Responsibility. However, because animals provide a better model of the complex physiological process of many diseases, replacement of animals in their entirety is often not possible. Therefore, the reduction of the number of animals required to predict a drug's efficacy and the refinement of the animal model to give robust predictions as to a drug's effects are important factors in drug screening models.

There are multiple aspects to take into account when performing the MEST test: the conventional and threshold experimental procedures, the factors affecting experimental data (laboratory conditions, administration vehicles and drug formulations, time after drug administration, and stimulus duration and site of stimulation) and the assessment of anticonvulsant activity. There lacks a method in which all the aforementioned factors are accounted for and which serves the purpose of quickly determining the efficacy of a compound in a clear-cut, binary way whilst requiring less total amounts of the test compound.

An object of the present invention is to provide a simple streamlined method to generate preliminary results of the anticonvulsive effect of test compounds, hereby named the mini-MEST test.

The present invention allows a lower number of animals to be used and in turn means the amount of test compound required for testing is not as high as for standard MEST tests. Further, due to the use of a logarithmic scale to increase the current of the electroshock, the present method is capable of providing a clear indication for the selection of candidate compounds during preclinical assessment, thus providing a more streamlined approach.

Such a method has been demonstrated to provide robust results in an effective manner as set out herein.

### BRIEF SUMMARY OF THE DISCLOSURE

In accordance with a first aspect of the present invention there is provided a method of assessing the pro- or anti-convulsant properties of compounds comprising the following steps:
a. dosing a number of animals with one of either vehicle, test compound or positive control compound;
b. individually assessing the treated animals for the production of a tonic hind limb extensor convulsion at a defined period of time post-dose from a single electroshock at a defined current;
c. decreasing or increasing the defined current if the preceding animal did or did not show tonic hind limb extensor convulsion, respectively and
d. collecting CC₅₀ values for the treated animals;
   characterised in that the number of animals used is at least 6.

Preferably the number of animals used is no more than 11.

Preferably the current of the electroshock is decreased or increased in a logarithmic scale.

Preferably the defined period of time post-dose is at least 15 minutes. More preferably, the defined period of time post-dose is 30 minutes. Most preferably, the defined period of time post-dose is 120 minutes.

Preferably the positive control compound is diazepam.

Alternatively, the positive control compound is sodium valproate.

Preferably the animal used is a mouse.

Alternatively, the animal used is a rat.

Alternatively, the animal used is pig.

### BRIEF DESCRIPTION OF THE DRAWINGS

Embodiments of the invention are further described hereinafter with reference to the accompanying drawings, in which:

FIG. 1 shows the effect of the Compound 1, as shown as Formula I, in the mini-MEST test in the mouse as described in Example 1.

FIG. 2 shows the effect of Compound 2, as shown as Formula II, in the mini-MEST test in the mouse as described in Example 2.

FIG. 3 shows the effect of Compound 3, as shown as Formula III, in the mini-MEST test in the mouse as described in Example 3.

FIG. 4 shows the effect of Compound 3, as shown as Formula III, in the MEST test in the mouse as described in Example 4.

### DEFINITIONS

“Cannabinoids” are a group of compounds including the endocannabinoids, the phytocannabinoids and those which are neither endocannabinoids or phytocannabinoids, hereinafter “syntho-cannabinoids”.

“Endocannabinoids” are endogenous cannabinoids, which are high affinity ligands of CB1 and CB2 receptors.

“Phytocannabinoids” are cannabinoids that originate in nature and can be found in the cannabis plant. The phytocannabinoids can be present in an extract including a botanical drug substance, isolated, or reproduced synthetically.

“Syntho-cannabinoids” are those compounds that are not found endogenously or in the cannabis plant. Examples include WIN 55212 and rimonabant.

An “isolated phytocannabinoid” is one which has been extracted from the cannabis plant and purified to such an extent that all the additional components such as secondary and minor cannabinoids and the non-cannabinoid fraction have been removed.

A “synthetic cannabinoid” is one which has been produced by chemical synthesis. This term includes modifying an isolated phytocannabinoid, by, for example, forming a pharmaceutically acceptable salt thereof.

A “substantially pure” cannabinoid is defined as a cannabinoid which is present at greater than 95% (w/w) pure. More preferably greater than 96% (w/w) through 97% (w/w) thorough 98% (w/w) to 99% % (w/w) and greater.

### DETAILED DESCRIPTION OF THE INVENTION

The following Examples describe for the first time how the mini-MEST test was used to assess the anti-convulsant activity of the following CBD analogues, Compound 1 as shown as Formula I, Compound 2 as shown as Formula II, and Compound 3 as shown as Formula III.

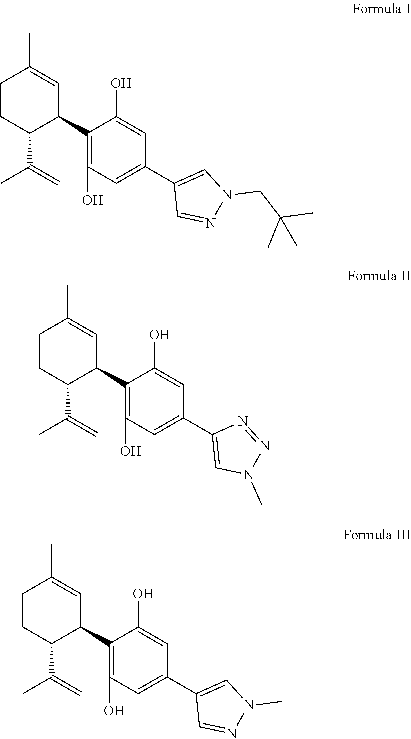 [SMILES: C=C(C)[C@@H]1CCC(C)=C[C@H]1c1c(O)cc(-c2cnn(CC(C)(C)C)c2)cc1O ; C=C(C)[C@@H]1CCC(C)=C[C@H]1c1c(O)cc(-c2cn(C)nn2)cc1O ; C=C(C)[C@@H]1CCC(C)=C[C@H]1c1c(O)cc(-c2cnn(C)c2)cc1O]

### Example 1: Evaluation of Cannabinoid Derivative for Anticonvulsant Activity Using the Maximal Electroshock Seizure Threshold (MEST) Test in the Mouse Using Minimal Sample Sizes (Mini MEST)

### Methods

### Study Details

Naïve mice were acclimatised to the procedure room in their home cages up to 7 days following arrival to the test facility, with food and water available ad libitum (see Table 1 for details).

All animals were weighed at the beginning of the study and assigned to treatment groups (n=6/group) based on a mean distribution of body weight across groups. All animals were dosed at 10 mL/kg via intraperitoneal (i.p.) injection, with either vehicle, test compound (50 mg/kg) or diazepam (2.5 mg/kg) (Tables 2 and 3 for details).

Animals were individually assessed for the production of a tonic hind limb extensor convulsion at 30 min post-dose for vehicle, test compound (50 mg/kg) or diazepam, from a single electroshock (see Table 4 for details). The first animal within a treatment group was given a shock at the expected or estimated CC₅₀ current. For subsequent animals, the current was lowered or raised depending on the convulsion outcome from the preceding animal in log scale intervals. Data generated from each treatment group were used to calculate the CC₅₀±SEM values for the treatment group (see Table 5 for details).

### Euthanasia and Sample Collection

Each animal was humanely killed immediately after production of a convulsion by destruction of the brain from striking the cranium, followed by confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in Lithium-heparin tubes and centrifuged at 4° C. for 10 min, at 1500×g. The resulting plasma was removed (>100 μL) and split into 2 aliquots stored in 0.5 mL Eppendorf tubes, containing 100 μL of ascorbic acid (100 mg/mL) for stabilisation. Brains were removed, washed in saline and halved. Each half was placed into separate 2 mL screw cap cryovials, weighed and frozen on cardice. Samples were stored at −80° C. until shipment.

### Animal Details

TABLE 1
Details of animal species, strain, sex,
order details and environmental conditions

| | |
|---|---|
| Species | Mouse |
| Strain | C57BL/6J |
| Sex | Male |
| No. of animals/group | n = 6/group |
| Weight range at study start | 20.8-24.8 g |
| Estimated age range at study | 8-9 weeks |
| start | |
| Environmental conditions | Housed in groups of 4-5, with standard |
| | conditions. |
| Lighting conditions | 12 h/2 h light cycle; 7 am lights on, 7 pm |
| | lights off; light intensity: |
| | 25-75 lux at bench level |
| Food and water | Food: Certified Rodent CR 14% Protein |
| | Rodent Diet, LabDiet ® 5CR4 |
| | Water: pathogen-free water from test |
| | facility |

### Compounds Details

TABLE 2
Details of Compound 1, batch, appearance, supplier, vehicle used for
formulation

| | |
|---|---|
| Storage conditions | Room temperature |
| Appearance | Cream powder |
| Vehicle used for formulation | 1:2:17 Ethanol: Kolliphor HS |
| | (Solutol): saline |

TABLE 3
Details of Diazepam, batch, appearance, supplier, vehicle used for
formulation

| | |
|---|---|
| Appearance | White powder |
| Vehicle used for formulation | 1:1:18 Ethanol: Kolliphor EL |
| | (Cremaphor): saline |

### Vehicle Preparation:

5% ethanol, 10% Kolliphor HS (Solutol) in 85% Saline solution

1 mL of Ethanol, 2 mL of Kolliphor HS (Solutol)—warmed to 60° C., in 17 mL of saline (1:2:17).

### Data Recorded and Analysis

TABLE 4
Details of data recorded in visual observations, mini MEST test, mini MEST data
analysis and statistical analysis.

| | |
|---|---|
| Visual observations | Animals were observed throughout the study from |
| | the start of dosing. Any abnormal signs were |
| | recorded and reported. |
| Mini MEST test | Mini MEST was run between 8 am to 4 pm under |
| | normal light conditions. |
| | Electroshock was delivered using a Hugo Sachs |
| | Electronik stimulator, with an adjustable constant |
| | current (1-300 mA). Electroshock duration is 0.1 |
| | seconds delivered via corneal electrodes on both |
| | eyes. |
| | Induction of seizure from the electroshock was |
| | measured as an all-or-nothing effect scored as |
| | either present (+) or absent (0) of tonic hind limb |
| | extensor convulsions for each animal. |
| ‘Up-and-down method’ based on | The current was lowered or raised in log 0.06:10^{∧} |
| Kimball AW et al., 1957 | (1 + x*0.06) mA intervals (see raw results in |
| | Appendix) if the preceding animal did or did not |
| | show tonic hind limb extension, respectively. If |
| | tonic hind limb extension was absent in the |
| | animal, the subsequent animal will receive a |
| | raised current level. If tonic hind limb |
| | extension was present in the animal, |
| | the subsequent animal will receive a |
| | lowered current level. This procedure was |
| | continued for all mice within a treatment group. |
| Mini MEST data analysis | The data for each treatment group were recorded |
| | as the number of +'s and 0's at each current level |
| | employed and this information was then used to |
| | calculate the CC₅₀ value (current required for 50% |
| | of the animals to show seizure behaviour) ± |
| | standard error of mean (SEM) based on Kimball et |
| | al. (1957). Test compound effects were also |
| | calculated as percentage change in CC₅₀ from the |
| | vehicle control group. |
| Statistical analysis | Significant difference between drug-treated |
| | animals and controls were assessed according to |
| | Litchfield and Wilcoxon (1949), using Microsoft |
| | Excel macro. |

### Results

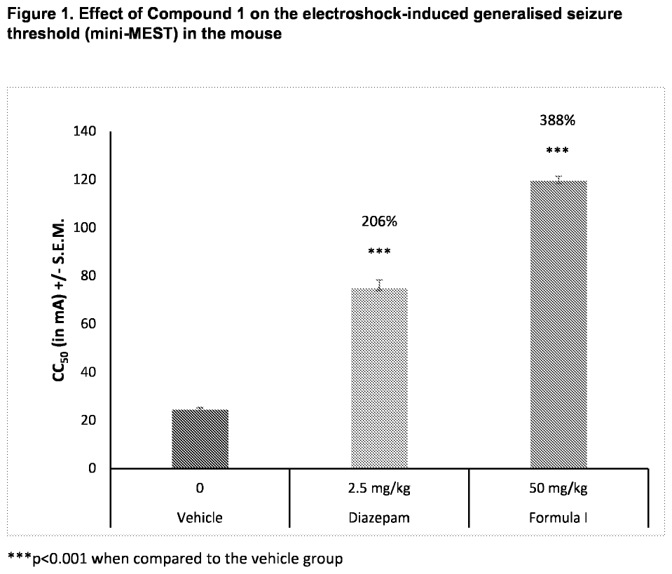

FIG. 1 and Table 5 describe the data produced in this experiment, and raw results are shown in the Appendix.

In the vehicle group, the CC₅₀ value was calculated to be 24.5 mA.

In the diazepam (2.5 mg/kg) treated group, administered i.p. 30 minutes before the test, the CC₅₀ value was 75.0 mA. This result was statistically significant (p<0.001) compared to the vehicle control.

In the test compound treatment group, administered i.p. 30 minutes before the test, the compound produced a statistically significant CC₅₀ value compared to vehicle, 119.5 mA.

Such data are indicative that this compound will be of therapeutic benefit.

TABLE 5
mini-MEST results table and statistical analysis.

| | | Test time | | | % change | |
|---|---|---|---|---|---|---|
| | Dose | post | | CC₅₀ ± | from | Signi- |
| Treatment | (mg/kg) | dose (min) | N | SEM | vehicle | ficance |
| Vehicle | 0 | 30 | 6 | 24.5 ± | — | — |
| | | | | 0.9 | | |
| Diazepam | 2.5 | 30 | 6 | 75.0 ± | 206% | P < 0.001 |
| | | | | 3.4 | | |
| Compound | 50 | 30 | 6 | 119.5 ± | 388% | P < 0.001 |
| 1 | | | | 1.9 | | |

### Conclusion

The positive control, diazepam (2.5 mg/kg) administered at 30 min post-dose (i.p.) produced a significant increase in seizure threshold. This result clearly demonstrates the robustness of the presently claimed method and validates the method used.

Compound 1 (50 mg/kg) administered at 30 min post-dose (i.p.) produced a produced a significant increase in seizure threshold, which suggests this compound exhibits anticonvulsive properties.

Thus, the mini-MEST method used was capable of providing a clear indication of the test compound's anticonvulsant properties.

### Example 2: Evaluation of Cannabinoid Derivative for Anticonvulsant Activity Using the Maximal Electroshock Seizure Threshold (MEST) Test in the Mouse Using Minimal Sample Sizes (Mini MEST)

The Example below was carried out similar to Example 1 outlined above using Compound 2 as according to Formula II.

### Methods

### Study Details

Naïve mice were acclimatised to the procedure room in their home cages up to 7 days following arrival to the test facility, with food and water available ad libitum (see Table 6 for details).

All animals were weighed at the beginning of the study and assigned to treatment groups (n=6/group) based on a mean distribution of body weight across groups. All animals were dosed at 10 mL/kg via intraperitoneal (i.p.) injection, with either vehicle, test compound (5 or 50 mg/kg) or diazepam (2.5 mg/kg) (Tables 7 and 8 for details).

Animals were individually assessed for the production of a tonic hind limb extensor convulsion at 30 min post-dose for vehicle, 15 and 30 minutes for test compound at 5 and 50 mg/kg respectively or diazepam, from a single electroshock (see Table 9 for details). The first animal within a treatment group was given a shock at the expected or estimated CC₅₀ current. For subsequent animals, the current was lowered or raised depending on the convulsion outcome from the preceding animal in log scale intervals. Data generated from each treatment group were used to calculate the CC₅₀±SEM values for the treatment group (see Table 10 for details).

### Euthanasia and Sample Collection

Each animal was humanely killed immediately after production of a convulsion by destruction of the brain from striking the cranium, followed by confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in Lithium-heparin tubes and centrifuged at 4° C. for 10 min, at 1500×g. The resulting plasma was removed (>100 μL) and split into 2 aliquots stored in 0.5 mL Eppendorf tubes, containing 100 μL of ascorbic acid (100 mg/mL) for stabilisation. Brains were removed, washed in saline and halved. Each half was placed into separate 2 mL screw cap cryovials, weighed and frozen on cardice. Samples were stored at −80° C. until shipment.

### Animal Details

TABLE 6
Details of animal species, strain, sex, order details and environmental
conditions

| | |
|---|---|
| Species | Mouse |
| Strain | C57BL/6J |
| Sex | Male |
| No. of animals /group | n = 6/group |
| Weight range at study start | 21.5-25.9 g |
| Estimated age range at study start | 8-9 weeks |
| Environmental conditions | Housed in groups of 4-5, with standard |
| | conditions. |
| Lighting conditions | 12 h/12 h light cycle; 7 am lights on, |
| | 7 pm lights off; light intensity: |
| | 25-75 lux at bench level |
| Food and water | Food: Certified Rodent CR 14% Protein |
| | Rodent Diet, LabDiet ® 5CR4 |
| | Water: pathogen-free water from test |
| | facility |

### Compounds Details

TABLE 7
Details of Compound 2, batch, appearance, supplier, vehicle used for
formulation

| | |
|---|---|
| Storage conditions | Room temperature |
| Appearance | White powder |
| Vehicle used for formulation | 1:2:17 Ethanol: Kolliphor HS |
| | (Solutol): saline |

TABLE 8
Details of Diazepam, batch, appearance, supplier, vehicle used for
formulation

| | |
|---|---|
| Appearance | White powder |
| Vehicle used for formulation | 1:1:18 Ethanol: Kolliphor EL |
| | (Cremaphor): saline |

Vehicle preparation:
5% ethanol, 10% Kolliphor HS (Solutiol) in 85% Saline solution
1 mL of Ethanol, 2 mL of Kolliphor HS (Solutol)-warmed to 60º C., in 17 mL of saline (1:2:17).

### Data Recorded and Analysis

TABLE 9
Details of data recorded in visual observations, mini MEST test, mini MEST data
analysis and statistical analysis.

| | |
|---|---|
| Visual observations | Animals were observed throughout the study from |
| | the start of dosing. Any abnormal signs were |
| | recorded and reported. |
| Mini MEST test | Mini MEST was run between 8 am to 4 pm under |
| | normal light conditions. |
| | Electroshock was delivered using a Hugo Sachs |
| | Electronik stimulator, with an adjustable constant |
| | current (1-300 mA). Electroshock duration is 0.1 |
| | seconds delivered via corneal electrodes on both |
| | eyes. |
| | Induction of seizure from the electroshock was |
| | measured as an all-or-nothing effect scored as |
| | either present (+) or absent (0) of tonic hind limb |
| | extensor convulsions for each animal. |
| ‘Up-and-down method’ based on | The current was lowered or raised in log 0.06:10^{∧} |
| Kimball AW et al., 1957 | (1 + x*0.06) mA intervals (see raw results in |
| | Appendix) if the preceding mouse did or did not |
| | show tonic hind limb extension, respectively. If |
| | tonic hind limb extension was absent in the |
| | animal, the subsequent animal will |
| | receive a raised current level. |
| | If tonic hind limb extension was present in |
| | the animal, the subsequent animal will receive a |
| | lowered current level. This procedure was |
| | continued for all rats within a treatment group. |
| Mini MEST data analysis | The data for each treatment group were recorded |
| | as the number of +'s and 0's at each current level |
| | employed and this information was then used to |
| | calculate the CC₅₀ value (current required for 50% |
| | of the animals to show seizure behaviour) ± |
| | standard error of mean (SEM) based on Kimball et |
| | al. (1957). Test compound effects were also |
| | calculated as percentage change in CC₅₀ from the |
| | vehicle control group. |
| Statistical analysis | Significant difference between drug-treated |
| | animals and controls were assessed according to |
| | Litchfield and Wilcoxon (1949), using Microsoft |
| | Excel macro. |

### Results

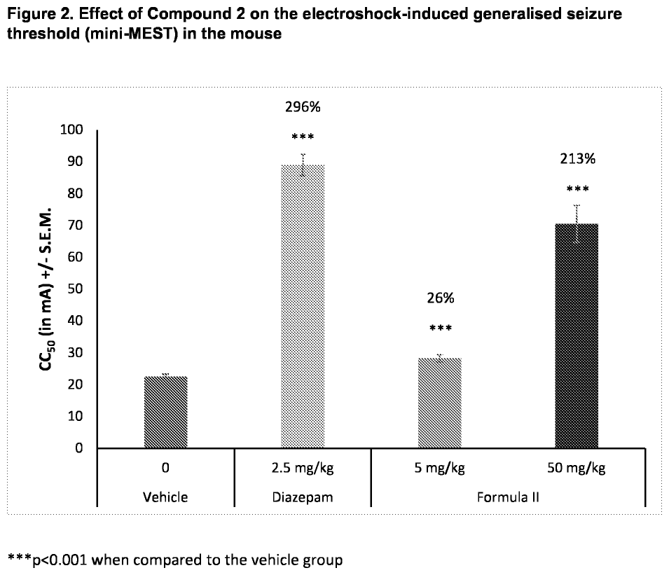

FIG. 2 and Table 10 describe the data produced in this experiment, and raw results are shown in the Appendix.

In the vehicle group, the CC₅₀ value was calculated to be 22.5 mA.

In the diazepam (2.5 mg/kg) treated group, administered i.p. 30 minutes before the test, the CC₅₀ value was 89.0 mA. This result was statistically significant (p<0.001) compared to the vehicle control.

In the test compound treatment groups, administered i.p. 15 and 30 minutes before the test, the compound at both doses produced statistically significant CC₅₀ values compared to vehicle.

Such data are indicative that this compound will be of therapeutic benefit.

TABLE 10
mini-MEST results table and statistical analysis.

| | | Test time | | | % change | |
|---|---|---|---|---|---|---|
| | Dose | post | | CC₅₀ ± | from | Signi- |
| Treatment | (mg/kg) | dose (min) | N | SEM | vehicle | ficance |
| Vehicle | 0 | 30 | 6 | 22.5 +/− 0.9 | — | — |
| Diazepam | 2.5 | 30 | 6 | 89.0 +/− 3.4 | 296% | P < 0.001 |
| Compound | 5 | 15 | 6 | 28.3 +/− 1.1 | 26% | P < 0.001 |
| 2 | | | | | | |
| Compound | 50 | 30 | 6 | 70.5 +/− 5.8 | 213% | P <0 .001 |
| 2 | | | | | | |

### Conclusion

The positive control, diazepam (2.5 mg/kg) administered at 30 min post-dose (i.p.) produced a significant increase in seizure threshold. This result clearly demonstrates the robustness of the presently claimed method and validates the method used.

Compound 2 tested at 5 & 50 mg/kg administered 15 and 30 mins respectively before testing (i.p.) produced a significant increase in seizure threshold as compared to vehicle, which suggests this compound exhibits anticonvulsive properties.

The data generated provides clear evidence of a dose-related increase in mini-MEST, further confirming the consistency of the method used.

The following example demonstrates the anti-convulsant activity for the CBD analogue, Compound 3 as shown as Formula III in the mini-MEST model. Additionally, data is provided from the same compound in the standard MEST model in example 4. Such data demonstrate the efficacy of the mini-MEST model in predicting the anti-convulsant effects of a test compound.

### Example 3: Evaluation of Cannabinoid Derivative for Anticonvulsant Activity Using the Maximal Electroshock Seizure Threshold (MEST) Test in the Mouse Using Minimal Sample Sizes (Mini MEST)

The Example below was carried out similarly to Examples 1 and 2 outlined above using Compound 3 as according to Formula III.

### Methods

### Study Details

Naïve mice were acclimatised to the procedure room in their home cages up to 7 days following arrival to the test facility, with food and water available ad libitum (see Table 11 for details).

All animals were weighed at the beginning of the study and assigned to treatment groups (n=6/group) based on a mean distribution of body weight across groups. All animals were dosed at 10 mL/kg via intraperitoneal (i.p.) injection, with either vehicle, test compound (200 mg/kg) or diazepam (2.5 mg/kg) (Tables 12 and 13 for details).

Animals were individually assessed for the production of a tonic hind limb extensor convulsion at 120 minutes post-dose for vehicle, 120 minutes for test compound and 30 minutes for diazepam, from a single electroshock (see Table 14 for details). The first animal within a treatment group was given a shock at the expected or estimated CC₅₀ current. For subsequent animals, the current was lowered or raised depending on the convulsion outcome from the preceding animal in log scale intervals. Data generated from each treatment group were used to calculate the CC₅₀±SEM values for the treatment group (see Table 15 for details).

### Euthanasia and Sample Collection

Each animal was humanely killed immediately after production of a convulsion by destruction of the brain from striking the cranium, followed by confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in Lithium-heparin tubes and centrifuged at 4° C. for 10 min, at 1500×g. The resulting plasma was removed (>100 μL) and split into 2 aliquots stored in 0.5 mL Eppendorf tubes, containing 100 μL of ascorbic acid (100 mg/mL) for stabilisation. Brains were removed, washed in saline and halved. Each half was placed into separate 2 mL screw cap cryovials, weighed and frozen on cardice. Samples were stored at −80° C. until shipment.

### Animal Details

TABLE 11
Details of animal species, strain, sex, order details and environmental
conditions

| | |
|---|---|
| Species | Mouse |
| Strain | C57BL/6J |
| Sex | Male |
| No. of animals/group | n = 6/group |
| Weight range at study start | 19.2-24.7 g |
| Estimated age range at study start | 8-9 weeks |
| Environmental conditions | Housed in groups of 4-5, with standard |
| | conditions. |
| Lighting conditions | 12 h/12h light cycle; 7 am lights on, |
| | 7pm lights off; light intensity: 25-75 |
| | lux at bench level |
| Food and water | Food: Certified Rodent CR 14% Protein |
| | Rodent Diet, LabDiet ® 5CR4 |
| | Water: pathogen-free water from test |
| | facility |

### Compounds Details

TABLE 12
Details of Compound 3, batch, appearance, supplier, vehicle used for
formulation

| | |
|---|---|
| Storage conditions | Room temperature |
| Appearance | White powder |
| Vehicle used for formulation | 1:2:17 Ethanol: Kolliphor HS |
| | (Solutol): saline |

TABLE 13
Details of Diazepam, batch, appearance, supplier, vehicle used for
formulation

| | |
|---|---|
| Appearance | White powder |
| Vehicle used for formulation | 1:1:18 Ethanol: Kolliphor EL |
| | (Cremaphor): saline |

### Vehicle Preparation:

5% ethanol, 10% Kolliphor HS (Solutiol) in 85% Saline solution

1 mL of Ethanol, 2 mL of Kolliphor HS (Solutol)—warmed to 60° C., in 17 mL of saline (1:2:17).

### Data Recorded and Analysis

TABLE 14
Details of data recorded in visual observations, mini MEST test, mini MEST data
analysis and statistical analysis.

| | |
|---|---|
| Visual observations | Animals were observed throughout the study from |
| | the start of dosing. Any abnormal signs were |
| | recorded and reported. |
| Mini MEST test | Mini MEST was run between 8 am to 4 pm under |
| | normal light conditions. |
| | Electroshock was delivered using a Hugo Sachs |
| | Electronik stimulator, with an adjustable constant |
| | current (1-300 mA). Electroshock duration is 0.1 |
| | seconds delivered via corneal electrodes on both |
| | eyes. |
| | Induction of seizure from the electroshock was |
| | measured as an all-or-nothing effect scored as |
| | either present (+) or absent (0) of tonic hind limb |
| | extensor convulsions for each animal. |
| ‘Up-and-down method’ based on | The current was lowered or raised in log 0.06:10^{∧} |
| Kimball AW et al., 1957 | (1 + x*0.06) mA intervals (see raw results in |
| | Appendix) if the preceding mouse did or did not |
| | show tonic hind limb extension, respectively. If |
| | tonic hind limb extension was absent in the |
| | animal, the subsequent animal |
| | will receive a raised current level. |
| | If tonic hind limb extension was present in |
| | the animal, the subsequent animal will receive a |
| | lowered current level. This procedure was |
| | continued for all rats within a treatment group. |
| Mini MEST data analysis | The data for each treatment group were recorded |
| | as the number of +'s and 0's at each current level |
| | employed and this information was then used to |
| | calculate the CC₅₀ value (current required for 50% |
| | of the animals to show seizure behaviour) ± |
| | standard error of mean (SEM) based on Kimball et |
| | al. (1957). Test compound effects were also |
| | calculated as percentage change in CC₅₀ from the |
| | vehicle control group. |
| Statistical analysis | Significant difference between drug-treated |
| | animals and controls were assessed according to |
| | Litchfield and Wilcoxon (1949), using Microsoft |
| | Excel macro. |

### Results

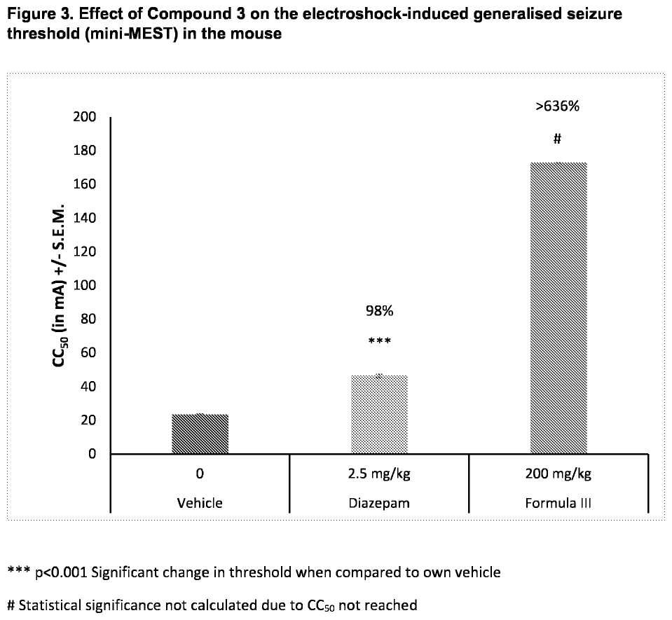

FIG. 3 and Table 15 describe the data produced in this experiment, and raw results are shown in the Appendix.

In the vehicle group, the CC₅₀ value was calculated to be 23.5 mA.

In the diazepam (2.5 mg/kg) treated group, administered i.p. 30 minutes before the test, the CC₅₀ value was 46.5 mA. This result was statistically significant (p<0.001) compared to the vehicle control.

In the test compound treatment group, administered i.p. 120 minutes before the test, the compound tested at 200 mg/kg produced a CC₅₀>173 mA; an exact value was not calculated as a “+” was not seen within the 6 animals tested. Although CC₅₀ was not determined and statistical significance was not achieved, the drug showed a clear increase in seizure threshold in the mini-MEST as compared to vehicle.

Such data are indicative that this compound will be of therapeutic benefit.

TABLE 15
mini-MEST results table and statistical analysis.

| | | Test time | | | | |
|---|---|---|---|---|---|---|
| | | post | | | % change | |
| | Dose | dose | | CC₅₀ ± | from | Signi- |
| Treatment | (mg/kg) | (min) | N | SEM | vehicle | ficance |
| Vehicle | 0 | 120 | 6 | 23.5 +/− 0.3 | — | — |
| Diazepam | 2.5 | 30 | 6 | 46.5 +/− 1.0 | 98% | P < 0.001 |
| Compound | 200 | 120 | 6 | >173 | >636% | # |
| 3 | | | | | | |

# Statistical significance not calculated due to CC₅₀ not reached

### Conclusion

The positive control, diazepam (2.5 mg/kg) administered at 30 min post-dose (i.p.) produced a significant increase in seizure threshold. This result clearly demonstrates the robustness of the presently claimed method and validates the method used.

Compound 3 tested at 200 mg/kg administered 120 mins before testing (i.p.) showed a clear increase in seizure threshold as compared to vehicle, which suggests this compound exhibits anticonvulsive properties.

The data generated using the mini-MEST method presents clear evidence of the potential of this compound as an anticonvulsant.

### Example 4: Evaluation of Cannabinoid Derivative for Anticonvulsant Activity Using the Maximal Electroshock Seizure Threshold (MEST) Test in the Mouse

The efficacy of Compound 3 was tested in a mouse model of generalised seizure, the maximal electroshock seizure threshold (MEST) test.

### Methods

### Study Details

Naïve mice were acclimatised to the procedure room in their home cages for up to 7 days, with food and water available ad libitum.

All animals were weighed at the beginning of the study and randomly assigned to treatment groups (n=12/group) based on a mean distribution of body weight across groups. All animals were dosed at 10 ml/kg via intraperitoneal (i.p) injection, with either vehicle, test compound at 2, 20 or 200 mg/kg or diazepam at 2.5 mg/kg.

Animals were individually assessed for the production of a tonic hind limb extensor convulsion at 30 min post-dose for vehicle, 30 min post-dose for test compound and 30 min post-dose for diazepam, from a single electroshock.

The first animal within a treatment group was given a shock at the expected or

estimated CC₅₀ current. For subsequent animals, the current was lowered or raised depending on the convulsions outcome from the preceding animal in 5 mA intervals.

Data generated from each treatment group were used to calculate the CC₅₀±SEM values for the treatment group.

### Test Compounds:

Vehicle: (5% ethanol, 10% solutol, 85% Saline) was prepared as follows: 1 mL of ethanol, 2 mL of solutol were warmed to 60° ° C., in 17 mL of saline (1:2:17).

Positive control: diazepam was used at 2.5 mg/kg.

The test compound used was Compound 3. Test compound was administered at 2, 20 and 200 mg/kg (i.p.) in a 1:2:17 ethanol:solutol:0.9% saline formulation.

### Sample Collection:

Each animal was humanely killed immediately after production of a convulsion by destruction of the brain from striking the cranium, followed by the confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in Lithium-heparin tubes and centrifuged at 4° C. for 10 minutes at 1500×g. The resulting plasma was removed (>100 μL) and split into 2 aliquots of 0.5 mL Eppendorf tubes containing 100 μL of ascorbic acid (100 mg/mL) for stabilisation. Brains were removed, washed in saline and halved. Each half was placed into separate 2 mL screw cap cryovials, weighed and frozen on cardice.

### Statistical Analysis

The data for each treatment group were recorded as the number of +'s and 0's at each current level employed and this information is then used to calculate the CC₅₀ value (current required for 50% of the animals to show seizure behaviour)±standard error.

Test compound effects were also calculated as percentage change in CC₅₀ from the vehicle control group.

Significant difference between drug-treated animals and controls were assessed according to Litchfield and Wilcoxon (1949).

### Results

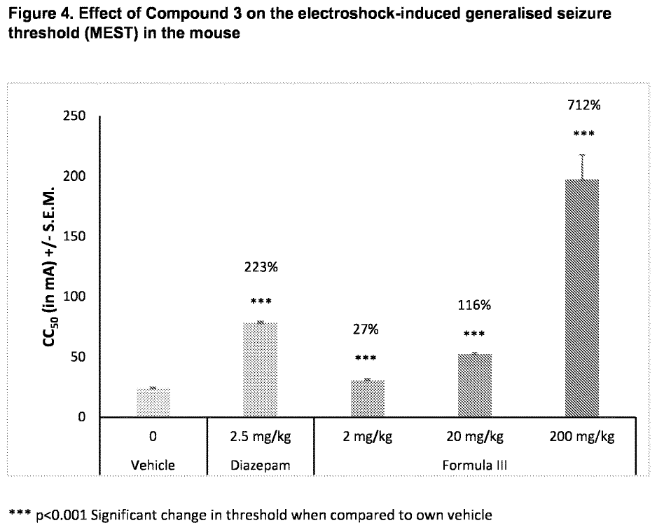

FIG. 4 and Table 16 describe the data produced in this experiment, and raw results are shown in the Appendix.

In the vehicle group, the CC₅₀ value was calculated to be 24.3 mA.

In the diazepam (2.5 mg/kg) treated group, administered i.p. 30 minutes before the test, the CC₅₀ value was 78.5 mA. This result was statistically significant (p<0.001) compared to the vehicle control. One animal in the diazepam group, was not dosed due to welfare issues from fighting.

In the test compound treatment groups, administered i.p. 30 minutes before the test, the compound produced a statistically significant CC₅₀ value compared to vehicle at all three doses of the compound.

TABLE 16
Evaluation of effect of Compound 3 in the MEST test

| | | Test time | | | | % change |
|---|---|---|---|---|---|---|
| | Dose | post dose | | CC₅₀ +/− | Signi- | from |
| Treatment | (mg/kg) | (min) | N | SEM | ficance | vehicle |
| Vehicle | — | 30 | 12 | 24.3 ± 0.4 | — | — |
| Diazepam | 2.5 | 30 | 11 | 78.5 ± 1.0 | P < 0.001 | 223% |
| Compound | 2 | 30 | 12 | 30.8 ± 1.0 | P < 0.001 | 27% |
| 3 | | | | | | |
| Compound | 20 | 30 | 12 | 52.5 ± 1.3 | P < 0.001 | 116% |
| 3 | | | | | | |
| Compound | 200 | 30 | 12 | 197.5 ± 20.4 | P < 0.001 | 712% |
| 3 | | | | | | |

### Conclusions

These data demonstrate a therapeutic effect for Compound 3 with a dose-related increase in MEST, which suggests that this compound exhibits anticonvulsive properties.

Thus, the data produced using the standard MEST model is consistent with the results of the mini-MEST model from Example 3 and reaffirms its conclusion. This consistency proves how the novel method of this application is able to generate robust results in an effective manner to be a useful predictor for the full MEST model.

Further, it has been shown that through the use of a logarithmic scale to increase or decrease the current, a smaller group of animals could be used in the mini-MEST method, thus achieving the overall aim of lowering number of animals and quantity of test compounds used.

### APPENDIX

Raw data for Example 1

| | Current | Animal no. | | | | | | | |
|---|---|---|---|---|---|---|---|---|---|
| Treatment: Vehicle | (mA) | 1 | 2 | 3 | 4 | 5 | 6 | +'s | 0's |
| Dose: — | 22 | | | 0 | | 0 | | 0 | 2 |
| Route: i.p. | 25 | | + | | + | | 0 | 2 | 1 |
| Predose: 30 mins | 29 | + | | | | | | 1 | 0 |
| | Current | Animal no. | | | | | | | |
| Treatment: Diazepam | (mA) | 7 | 8 | 9 | 10 | 11 | 12 | +'s | 0's |
| Dose: 2.5 mg/kg | 66 | 0 | | 0 | | | | 0 | 2 |
| Route: i.p. | 75 | | + | | 0 | | 0 | 1 | 2 |
| Predose: 30 mins | 87 | | | | | + | | 1 | 0 |
| | Current | Animal no. | | | | | | | |
| Treatment: Compound 1 | (mA) | 13 | 14 | 15 | 16 | 17 | 18 | +'s | 0's |
| Dose: 50 mg/kg | 87 | 0 | | | | | | 0 | 1 |
| Route: i.p. | 100 | | 0 | | | | | 0 | 1 |
| Predose: 30 mins | 114 | | | 0 | | 0 | | 0 | 2 |
| | 131 | | | | + | | 0 | 1 | 1 |

Raw data for Example 2

| Treatment: | Current | Animal no. | | | | | | | |
|---|---|---|---|---|---|---|---|---|---|
| Vehicle | (mA) | 1 | 2 | 3 | 4 | 5 | 6 | +'s | 0's |
| Dose: — | 19 | | | 0 | | | | 0 | 1 |
| Route: i.p. | 22 | | + | | 0 | | 0 | 1 | 2 |
| Predose: 30 mins | 25 | + | | | | + | | 2 | 0 |
| Treatment: | Current | Animal no. | | | | | | | |
| Diazepam | (mA) | 7 | 8 | 9 | 10 | 11 | 12 | +'s | 0's |
| Dose: 2.5 mg/kg | 75 | | 0 | | | | | 0 | 1 |
| Route: i.p. | 87 | + | | 0 | | 0 | | 1 | 2 |
| Predose: 30 mins | 100 | | | | + | | + | 2 | 0 |
| Treatment: | Current | Animal no. | | | | | | | |
| Compound 2 | (mA) | 13 | 14 | 15 | 16 | 17 | 18 | +'s | 0's |
| Dose: 5 mg/kg | 25 | | | 0 | | 0 | | 0 | 2 |
| Route: i.p. | 29 | | + | | + | | 0 | 2 | 1 |
| Predose: 15 mins | 33 | + | | | | | | 1 | 0 |
| Treatment: | Current | Animal no. | | | | | | | |
| Compound 2 | (mA) | 19 | 20 | 21 | 22 | 23 | 24 | +'s | 0's |
| Dose: 50 mg/kg | 57 | 0 | | | | | | 0 | 1 |
| Route: i.p. | 66 | | 0 | | | | + | 1 | 1 |
| Predose: 30 mins | 75 | | | 0 | | + | | 1 | 1 |
| | 87 | | | | + | | | 1 | 0 |

Raw data for Example 3

| | Current | Animal no. | | | | | | | |
|---|---|---|---|---|---|---|---|---|---|
| Treatment: Vehicle | (mA) | 1 | 2 | 3 | 4 | 5 | 6 | +'s | 0's |
| Dose: — | 22 | 0 | | 0 | | 0 | | 0 | 3 |
| Route: i.p. | 25 | | + | | + | | + | 3 | 0 |
| Predose: 120 mins | | | | | | | | | |
| | Current | Animal no. | | | | | | | |
| Treatment: Diazepam | (mA) | 7 | 8 | 9 | 10 | 11 | 12 | +'s | 0's |
| Dose: 2.5 mg/kg | 43 | | 0 | | | | | 0 | 1 |
| Route: i.p. | 50 | + | | 0 | | | | 1 | 1 |
| Predose: 30 mins | 57 | | | | 0 | | | 0 | 1 |
| | 66 | | | | | 0 | | 0 | 1 |
| | 75 | | | | | | 0 | 0 | 1 |
| | Current | Animal no. | | | | | | +'s | 0's |
| Treatment: Compound 3 | (mA) | 13 | 14 | 15 | 16 | 17 | 18 | | |
| Dose: 200 mg/kg | 87 | 0 | | | | | | 0 | 1 |
| Route: i.p. | 100 | | 0 | | | | | 0 | 1 |
| Predose: 120 mins | 114 | | | 0 | | | | 0 | 1 |
| | 131 | | | | 0 | | | 0 | 1 |
| | 151 | | | | | 0 | | 0 | 1 |
| | 173 | | | | | | 0 | 0 | 1 |

Raw data for Example 4

| | Current | Animal no. | | | | | | | | | | | | | |
|---|---|---|---|---|---|---|---|---|---|---|---|---|---|---|---|
| Treatment: Vehicle | (mA) | 1 | 2 | 3 | 4 | 5 | 6 | 7 | 8 | 9 | 10 | 11 | 12 | +'s | 0's |
| Dose: — | 22 | | 0 | | 0 | | | | | | | | | 0 | 2 |
| Route: i.p. | 24 | + | | + | | 0 | | 0 | | 0 | | 0 | | 2 | 4 |
| Predose: 30 mins | 26 | | | | | | + | | + | | + | | + | 4 | 0 |
| | Current | Animal no. | | | | | | | | | | | | | |
| Treatment: Diazepam | (mA) | 13 | 14 | 15 | 16 | 17 | 18 | 19 | 20 | 21 | 22 | 23 | 24 | +'s | 0's |
| Dose: 2.5 mg/kg | 75 | 0 | | | | 0 | | 0 | | 0 | | 0 | | 0 | 5 |
| Route: i.p. | 80 | | 0 | | + | | + | | + | | + | | | 4 | 1 |
| Predose: 30 mins | 85 | | | + | | | | | | | | | | 1 | 0 |
| | Current | Animal no. | | | | | | | | | | | | | |
| Treatment: Compound 3 | (mA) | 25 | 26 | 27 | 28 | 29 | 30 | 31 | 32 | 33 | 34 | 35 | 36 | +'s | 0's |
| Dose: 2 mg/kg | 25 | | | | 0 | | | | 0 | | | | | 0 | 2 |
| Route: i.p. | 30 | 0 | | + | | 0 | | + | | 0 | | 0 | | 2 | 4 |
| Predose: 30 mins | 35 | | + | | | | + | | | | + | | + | 4 | 0 |
| | Current | Animal no. | | | | | | | | | | | | | |
| Treatment: Compound 3 | (mA) | 37 | 38 | 39 | 40 | 41 | 42 | 43 | 44 | 45 | 46 | 47 | 48 | +'s | 0's |
| Dose: 20 mg/kg | 45 | | | | | | | | | | 0 | | | 0 | 1 |
| Route: i.p. | 50 | | | 0 | | 0 | | 0 | | + | | 0 | | 1 | 4 |
| Predose: 30 mins | 55 | | + | | + | | + | | + | | | | 0 | 4 | 1 |
| | 60 | + | | | | | | | | | | | | 1 | 0 |
| | Current | Animal no. | | | | | | | | | | | | | |
| Treatment: Compound 3 | (mA) | 49 | 50 | 51 | 52 | 53 | 54 | 55 | 56 | 57 | 58 | 59 | 60 | +'s | 0's |
| Dose: 200 mg/kg | 180 | 0 | | 0 | | | | | | | | | | 0 | 2 |
| Route: i.p. | 185 | | + | | 0 | | | | | | | | | 1 | 1 |
| Predose: 30 mins | 190 | | | | | 0 | | | | | | | | 0 | 1 |
| | 195 | | | | | | 0 | | | | | | | 0 | 1 |
| | 200 | | | | | | | 0 | | 0 | | | | 0 | 2 |
| | 205 | | | | | | | | + | | 0 | | 0 | 1 | 2 |
| | 210 | | | | | | | | | | | + | | 1 | 0 |

1. A method of assessing the pro- or anti-convulsant properties of a test compound comprising the following steps:
a. dosing a number of animals with one of either a vehicle, the test compound or a positive control compound;
b. determining if the animals produce a tonic hind limb extensor convulsion at a defined period of time post-dose from a single electroshock at a defined current;
c. decreasing or increasing the defined current if the preceding animal did or did not produce tonic hind limb extensor convulsion, respectively and
d. collecting CC₅₀ values for the treated animals;
characterized in that the number of animals used is at least 6.

2. The method according to claim 1, wherein the number of animals used is no more than 11.

3. The method according to claim 1, wherein the current of the electroshock is decreased or increased in a logarithmic scale.

4. The method according to claim 1, wherein the defined period of time post-dose is at least 15 minutes.

5. The method according to claim 1, wherein the defined period of time post-dose is about 30 minutes.

6. The method according to claim 1, wherein the defined period of time post-dose is 120 minutes.

7. The method according to claim 1, wherein the positive control compound is diazepam.

8. The method according to claim 1, wherein the positive control compound is sodium valproate.

9. The method according to claim 1, wherein the animal used is a mouse.

10. The method according to claim 1, wherein the animal used is a rat.

11. The method according to claim 1, wherein the animal used is a pig.
